(19) United States
(12) Patent Application Publication
(10) Pub. No.: US 2012/0134943 A1
(43) Pub. Date: May 31, 2012

(54) Topical Compositions Comprising An Alkoxylated Diphenylacrylate Compound And An Aryl Carboxylic Ester

(76) Inventors: Ismail Ahmed Syed, Glen Oaks, NY (US); Milanka Susak, North York (CA); Linda Josephine Najdek, East Islip, NY (US); Mirela Cristina Ionita-Manzatu, Old Bethpage, NY (US)

(21) Appl. No.: 13/266,439

(22) PCT Filed: Apr. 28, 2010
(86) PCT No.: PCT/US10/32702

§ 371 Date: Feb. 6, 2012

Related U.S. Application Data
(60) Provisional application No. 61/176,515, filed on May 8, 2009.

(51) Int. Cl. A61K 8/40; A61K 8/66; A61Q 17/04

(52) U.S. Cl. 424/60; 514/785; 424/94.1

(57) ABSTRACT

A topical composition containing an alkoxylated diphenylacrylate compound and an aryl carboxylic ester is provided. Preferably, the topical composition is a sunscreen composition containing α-ethylhexyl α-cyano-β-(4-methoxyphenyl)-β-phenylacrylate and 2-phenylethyl benzoate, and optionally further containing 4,4′-t-butyl methoxydibenzoylmethane, which is characterized by improved photo-protection of the skin and is effective in preventing/reducing photo-damage of the skin upon exposure to sunlight or other sources of light in the ultraviolet (UV), visible, and infrared (IR) ranges.

### CROSS-REFERENCE TO RELATED APPLICATIONS

The present application claims priority of U.S. Provisional Application No. 61/176,515 filed on May 8, 2009.

### FIELD OF THE INVENTION

The invention relates to topical compositions with skin care or cosmetic properties. More particularly, the invention relates to topical compositions containing an alkoxylated diphenylacrylate compound in combination with an aryl carboxylic ester, and more specifically an ester of an aryl carboxylic acid and an arylalkyl alcohol.

### BACKGROUND OF THE INVENTION

There are many problems in formulating topical compositions. In order to be commercially acceptable, the composition must be stable. No matter how efficacious a product is, the product will not be commercially acceptable if it separates or otherwise deteriorates under normal shipping and storage conditions prior to sale. In addition, when compositions applied to the skin are meant to form a film, it is important that the film formed be relatively homogeneous on the skin. One criterion for assessing the adequacy of a film formed on skin is spreadability, which means that when a composition is applied to skin it should spread onto the skin to form a relatively even film. This is particularly important when it comes to color cosmetics and sunscreens. Obviously a sun bather does not want to apply a sunscreen film that leaves gaps in coverage, nor does a consumer want to apply a lipstick that does not evenly color the lips. It is believed that spreadability and relatively homogeneous film formation on skin is improved when the composition is formulated with ingredients that improve dispersibility of various components such as particulates, oils, and polymers, in the composition. Another formulation concern is aesthetics. No matter how efficacious a product is, consumers will not buy it if it is aesthetically unpleasant when applied to skin.

Diphenylacrylates are generally known for enhancing SPF when incorporated into sunscreens. U.S. Patent Publication 2009/0039323A1 teaches that certain α-cyanodiphenylacrylates act by accepting the triplet excited state energy generated by organic sunscreens such as Avobenzone when it is exposed to certain environmental conditions. The end result is that the excess energy formed by the unstable Avobenzone is then dissipated and does not result in an unstable ingredient that compromises the stability of the formula. However, cosmetic formulators must combine more than diphenylacrylates and organic sunscreens to make a commercially acceptable cosmetic formula, and it is very difficult to form a cosmetic composition with good spreadability, stability and aesthetic appeal when multiple ingredients of different polarity and hydrophobicity/hydrophilicity are combined. Such difficulty is particularly elevated when physical or inorganic sunscreen ingredients are included, due to their insolubility in both polar and non-polar solvents and their tendency to coagulate and clumps that may potentially clog the pores.

It has been found that in addition to their SPF enhancing capability, certain alkoxylated diphenylacrylates, particularly alkoxylated α-cyanodiphenylacrylates, are excellent dispersants for cosmetic ingredients. Despite their lipophilic character, alkoxylated α-cyanodiphenylacrylates can facilitate dispersion of cosmetic ingredients that are charged or polar or contain both lipophilic and hydrophilic moieties to form an easily spreadable topical cosmetic product. Most unexpectedly, when combined with an aryl carboxylic ester, the alkoxylated α-cyanodiphenylacrylates form a stable base that is particularly effective in solubilizing or dispersing organic and/or inorganic sunscreens, thereby enabling formation of stable sunscreen products of improved SPF protection, more aesthetic appeal, and extended shelf life.

### SUMMARY OF THE INVENTION

The present invention provides a topical composition comprising an alkoxylated diphenylacrylate compound and an aryl carboxylic ester in a pharmaceutically or cosmetically acceptable carrier. It has been discovered that the alkoxylated diphenylacrylate compound, when combined with the aryl carboxylic ester, is surprisingly and unexpectedly effective in solubilizing or dispersing cosmetic ingredients, including but not limited to organic and/or inorganic sunscreen actives.

More specifically, the present invention provides a topical composition comprising an alkoxylated α-cyanodiphenylacrylate compound and an ester of an aryl carboxylic acid and an arylalkyl alcohol in a pharmaceutically or cosmetically acceptable carrier. The alkoxylated α-cyanodiphenylacrylate compound preferably has the formula (I):

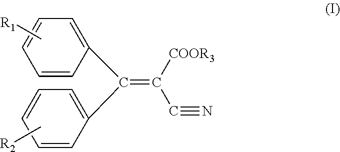

wherein one or both of R₁ and R₂ is a straight or branched-chain C₁-C₃₀ alkoxy radical, any non-alkoxy radical R₁ or R₂ is hydrogen, and R₃ is a straight or branched-chain C₁-C₃₀ alkyl radical. The ester of an aryl carboxylic acid and an arylalkyl alcohol as mentioned hereinabove preferably has the formula (II):

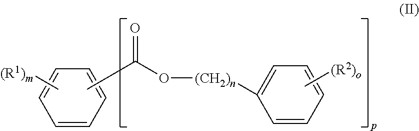

wherein R¹ and R² may be identical or different and are selected from the group consisting of a hydroxy group, a linear or branched C₁-C₄ alkyl or alkoxy radical, a nitro radical, an amino radical, and a C₆H₆SO₂ radical; m is an integer ranging from 0 to 5; n is an integer ranging from 1 to 10; o is an integer ranging from 0 to 5; p is an integer ranging from 1 to 6, provided that m+p<=6.

Other aspects and objectives of the present invention will become more apparent from the ensuing description, examples, and claims.

### DETAILED DESCRIPTION OF THE INVENTION, AND THE PREFERRED EMBODIMENTS THEREOF

Organic compounds containing one or more α-cyanodiphenylacrylate moieties, such as 2-ethylhexyl 2-cyano-3,3-diphenylacrylate (hereinafter “Octocrylene”), have long been used in combination with other organic sunscreen agents, such as butyl methoxydibenzoylmethane (hereinafter “Avobenzone”) and/or octyl methoxycinnamate (hereinafter “OMC”), to form sunscreen compositions. Although Avobenzone is an effective UV-A absorber, it is know to be unstable upon prolonged exposure to UV radiation, especially when combined with UV-B absorbers such as OMC. The α-cyanodiphenylacrylate compounds are known for improving the photo-stability of Avobenzone and therefore are typically used in combination therewith.

The inventors of the present invention discovered that a very specific group of α-cyanodiphenylacrylate compounds, namely the alkoxylated α-cyanodiphenylacrylate compounds, are surprisingly and unexpectedly effective in dissolving or dispersing cosmetic ingredients when combined with an aryl carboxylic ester, particularly an ester of an aryl carboxylic acid and an arylalkyl alcohol. Such a combination as described hereinabove provides a stable base formula that can be used to form different cosmetic or skin care products by incorporating various active ingredients. Such a combination is particularly suitable for solubilizing or dispersing organic and/or inorganic sunscreen agents, thereby forming stable sunscreen compositions characterized by boosted SPF, enhanced spreadability, better aesthetic appeal, and longer shelf life.

Although all alkoxylated α-cyanodiphenylacrylate compounds are suitable for the practice of the present invention, preferred alkoxylated α-cyanodiphenylacrylate compounds for use in the present invention are those with an alkoxyl substitution group at the para position of at least one of the phenyl rings, i.e., those represented by formula (III):

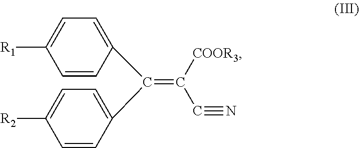

wherein one or both of R₁ and R₂ is a straight or branched-chain C₁-C₃₀ alkoxy radical, more preferably a straight or branched-chain C₁-C₈ alkoxy radical, any non-alkoxy radical R₁ or R₂ is hydrogen, and R₃ is a straight or branched-chain C₁-C₃₀ alkyl radical. Among straight or branched-chain C₁-C₈ alkoxy radicals, preferred are methoxy, ethoxy, n-propoxy, iospropoxy, n-butyoxy, isobutyoxy, and tert-butoxy radicals. Among straight or branched-chain C₁-C₃₀ alkyl radicals, preferred are methyl, ethyl, n-propyl, isopropyl, n-butyl, isobutyl, tert-butyl, n-amyl, isoamyl, neopentyl, n-hexyl, n-heptyl, n-octyl, 2-ethylhexyl, decyl and lauryl radicals. More preferred species of alkoxylated α-cyanodiphenylacrylate compounds include those having the formulas (IV)-(VI):

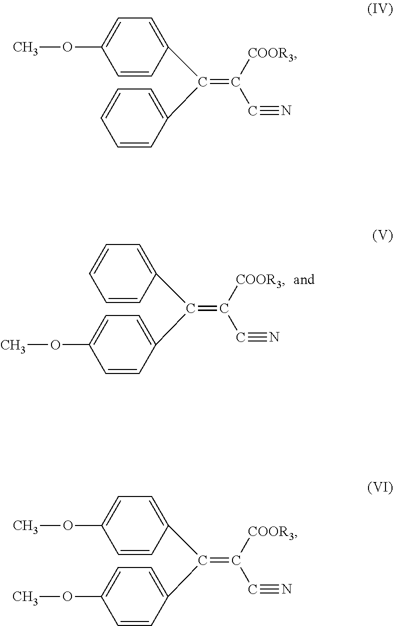

wherein R₃ is as previously defined.

Most preferred species of alkoxylated α-cyanodiphenylacrylate compound for the practice of the present invention is α-ethylhexyl α-cyano-β-(4-methoxyphenyl)-β-phenylacrylate (hereinafter “Methoxycrylene”), which is commercially available from HallStar Company (Chicago, Ill.). For more details about Methoxycrylene, see U.S. Patent Application Publication Nos. US2009/039322A, US2009/039323A, and US2009/042312A, as well as International Patent Application Publication No. WO09/020,675A, which are hereby incorporated by reference in their entirety for all purposes. Methoxycrylene has been used for stabilizing certain organic sunscreen agents that are susceptible to photo-decomposition upon exposure to UV radiation, such as Avobenzone and OMC. However, it has been discovered by the inventors that Methoxycrylene is a surprisingly and unexpectedly effective dispersing agent. When combined with an aryl carboxylic ester, Methoxycrylene forms a very stable base formula into which various cosmetic and skin care actives can be readily incorporated to form a wide range of cosmetic and skin care products. Such a base formula is especially effectively in solubilizing or dispersing organic and inorganic sunscreen agents, thereby forming cosmetic or sunscreen compositions of excellent spreadability and stability.

The alkoxylated diphenylacrylate compound can be present in the topical composition of the present invention in an amount ranging from about 0.01% to about 50%, preferably from about 0.1% to about 20%, and more preferably from about 1% to about 15%, by total weight of the composition. All percentages as described herein, unless otherwise specified, refer to the weight percentages calculated based on the total weight of the final composition. Aryl carboxylic esters suitable for the practice of the present invention include those formed by esterification of an aryl carboxylic acid with an arylalkyl alcohol, and preferably including those having the formula (II):

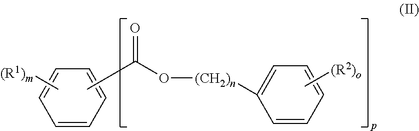

wherein R¹ and R² may be identical or different and are selected from the group consisting of a hydroxy group, a linear or branched C₁-C₄ alkyl or alkoxy radical, a nitro radical, an amino radical, and a C₆H₆SO₂ radical; m is an integer ranging from 0 to 5; n is an integer ranging from 1 to 10; o is an integer ranging from 0 to 5; p is an integer ranging from 1 to 6, provided that m+p<=6.

Among the aryl carboxylic esters described hereinabove, particularly preferred are 2-phenylethyl benzoate, 2-phenylethyl toluate, and di-2-phenylethyl phthalate, and most preferred is 2-phenylethyl benzoate having the formula:

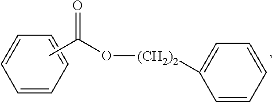

which is commercially available from International Special Products, Inc. (Wayne, N.J.) under the trade name X-Tend 226®.

The aryl carboxylic ester in accordance with the present invention may be present in an amount ranging from about 0.01% to about 50%, preferably from about 0.1% to about 20%, and more preferably from about 1% to about 15%, by total weight of the composition.

The topical composition of the present invention may further contain at least one dibenzoylmethane UV-screening agent, which can be effectively solubilized and stabilized by the base formula containing the alkoxylated α-cyanodiphenylacrylate and the aryl carboxylic ester as described hereinabove, thereby forming a sunscreen composition with improved photo-protection and extended protection duration. Preferred dibenzoylmethane compounds for the practice of the present invention include, but are not limited to: 2-methyldibenzoylmethane; 4-methyldibenzoylmethane; 4-isopropyldibenzoylmethane; 4-tert-butyldibenzoylmethane; 2,4-dimethyldibenzoylmethane; 2,5-dimethyldibenzoylmethane; 4,4′-diisopropyldibenzoylmethane; 4,4′-dimethoxydibenzoylmethane; 4-tert-butyl-4′-methoxydibenzoylmethane; 2-methyl-5-isopropyl-4′-methoxydibenzoylmethane; 2-methyl-5-tert-butyl-4′-methoxydibenzoylmethane; 2,4-dimethyl-4′-methoxydibenzoylmethane; and 2,6-dimethyl-4-tert-butyl-4′-methoxydibenzoylmethane. More preferred specifies of dibenzoylmethane compounds are 4-isopropyldibenzoylmethane, which is commercially available from Merck under the trade name “Eusolex 8020,” and 4-(tert-butyl)-4′-methoxydibenzoylmethane, which is commonly referred to as “Avobenzone” and is commercially available from Roche Vitamins under the trademark “Parsol 1789.”

If present, the dibenzoylmethane compound may be provided in an amount ranging from 0.01% to 30%, more preferably 0.1% to 15%, and most preferably from 1% to 8%, by total weight of the composition.

The topical composition of the present invention may further contain one or more additional organic sunscreen agents (i.e., other than the alkoxylated diphenylacrylate compound or the dibenzoylmethane compound), thereby providing sunscreen compositions of high SPF values (e.g., SPF measurements of greater than 25, 30, 35, 40, 45, or even 50 or more). If present, such additional organic sunscreen agents may each range from about 0.01 to 45% by weight of the total composition.

Exemplary organic sunscreen agents that can be used in combination with the inorganic particulate material and the alkoxylated diphenylacrylate compound of the present invention include, but are not limited to UVA and UVB sunscreens, such as benzophenones and derivatives thereof (e.g., benzophenone-3, dioxybenzone, sulisobenzone, octabenzone, hydroxy- and/or methoxy-substituted benzophenones, and benzophenonesulfonic acids and salts thereof); salicylic acid derivatives (e.g., ethylene glycol salicylate, triethanolamine salicylate, octyl salicylate, homomethyl salicylate, and phenyl salicylate); urocanic acid and derivatives thereof (e.g., ethyl urocanate); p-aminobenzoic acid (PABA) and derivatives thereof (e.g., ethyl/isobutyl/glyceryl esters thereof and 2-ethylhexyl p-dimethylaminobenzoate, which is also referred to as octyldimethyl PABA); anthranilates and derivatives thereof (e.g., o-amino-benzoates and various esters of amino-benzoic acid); benzalmalonate derivatives; benzimidazole derivatives; imidazolines; bis-benzazolyl derivatives; benzoazole/benzodiazole/benzotriazoles and derivatives thereof (e.g., 2-(2-hydroxy-5-methylphenyl)benzotriazole and methylene bis-benzotriazolyl tetramethylbutylphenol, which is commonly referred to as “Tinosorb M”); diesters or polyesters containing diphenylmethylene or 9H-fluorene substitutional groups; 2-phenyl-benzimidazole-5-sulphonic acid (PBSA); 4,4-diarylbutadienes; cinnamates and derivatives thereof (e.g., 2-ethylhexyl-p-methoxycinnamate, octyl-p-methoxycinnamate, umbelliferone, methylumbelliferone, methylaceto-umbelliferone, esculetin, methylesculetin, and daphnetin); camphors and derivatives thereof (e.g., 3-benzylidenecamphor, 4-methylbenzylidenecamphor, polyacrylamidomethyl benzylidenecamphor, benzylidene camphor sulfonic acid, and terephthalylidene dicamphor sulfonic acid, which is commonly referred to as “Encamsule”); triazines and derivatives thereof (e.g., 2,4-bis-{[4-(2-ethyl-hexyloxy)-2-hydroxy]-phenyl}-6-(4-methoxyphenyl)-1,3,5-triazine, which is commonly referred to as “Tinosorb S”); naphthalates and derivatives thereof (e.g., diethylhexyl-2,6-naphthalate); naphtholsulfonates and derivatives thereof (e.g., sodium salts of 2-naphthol-3,6-disulfonic and 2-naphthol-6,8-disulfonic acids); dibenzalacetone and benzalacetonephenone; diphenylbutadienes and derivatives thereof; di-hydroxynaphthoic acid and salts thereof; o- and p-hydroxybiphenyldisulfonates; coumarin derivatives (e.g., 7-hydroxy, 7-methyl, and 3-phenyl derivatives thereof); azoles/diazoles/triazoles and derivatives thereof (e.g., 2-acetyl-3-bromoindazole, phenyl benzoxazole, methyl naphthoxazole, and various aryl benzotriazoles); quinine and derivatives thereof (e.g., bisulfate, sulfate, chloride, oleate, and tannate salts thereof); quinoline and derivatives thereof (e.g., 2-phenylquinoline and 8-hydroxyquinoline salts); tannic acid and derivatives thereof (e.g., hexaethylether derivatives thereof); hydroquinone and derivatives thereof; uric acid and derivatives thereof; vilouric acid and derivatives thereof, and mixtures or combinations thereof. Salts and otherwise neutralized forms of certain acidic sunscreens from the list hereinabove are also useful herein. These organic sunscreen agents may be used alone or in combination of two or more. In addition, other known animal or vegetable extracts having UV light-absorbing ability may properly be used alone or in combination.

Organic sunscreen agents that are particularly useful for the practice of the present invention are: 2-ethylhexylsalicylate, 3,3,5-trimethylcyclohexylsalicylate, 2-ethylhexyl p-methoxycinnamate, 2-hydroxy-4-methoxybenzophenone, 2,2-dihydroxy-4-methoxybenzophenone, 2,4-bis-{4-(2-ethyl-hexyloxy)-2-hydroxyl-phenyl}-6-(4-methoxyphenyl)-1,3,5-triazine, methylene bis-benzotriazolyl tetramethylbutylphenol, terephthalylidene dicamphor sulfonic acid, diethylhexyl 2,6-naphthalate, digalloyltrioleate, ethyl 4-[bis(hydroxypropyl)]aminobenzoate, glycerol p-aminobenzoate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid, and mixtures or combinations thereof.

The topical composition of the present invention may include one or more inorganic particulate materials, which include, but are not limited to: titanium dioxide, zinc oxide, iron oxides, mica, titanated mica, alumina, talc, pearl powder, calcium carbonate, calcium phosphate, calcium silicate, calcium sulfate (gypsum), magnesium carbonate, magnesium oxide, magnesium silicate, magnesium aluminum silicate, magnesium trisilicate, aluminum silicate, aluminum magnesium silicate, silica, fumed silica, hydrated silica, spherical silica, silica silylate, bismuth oxychloride, chalk, kaolin, diatomaceous earth, fuller's earth, kaolin, sericite, muscovite, phlogopite, lepidolite, biotite, vermiculite, smectite clays, strontium silicate, metal tungstate, magnesium, silica alumina, zeolite, tin oxide, barium sulfate, fluorine apatite, hydroxyapatite, ceramic powder, colloidal silicone dioxide, boron nitride, hectorite, montmorillonite, bentonite, attapulgite, titanium hydroxide, trimagnesium phosphate, and the like. The inventors of the present invention discovered that the alkoxylated α-cyanodiphenylacrylate compounds are particularly effective in facilitating the dispersion of inorganic particulates and improving the overall aesthetical appeal of the resulting topical composition. This discovery is particularly surprising and unexpected in light of the fact that the inorganic particulates, such as metal oxides, are highly charged particles, while the alkoxylated α-cyanodiphenylacrylate compounds are lipophilic organic compounds. When the inorganic particulates are inorganic sunscreen agents, such as titanium dioxide and zinc oxide, the combination thereof with the alkoxylated α-cyanodiphenylacrylate compound surprisingly and unexpectedly boosts the SPF of the overall composition, which is believed due to the superior dispersing properties of the alkoxylated α-cyanodiphenylacrylate compound.

Preferred inorganic particulate materials are selected from metal oxides, such as titanium dioxide, zinc oxide, iron oxides, magnesium oxide, alumina (or aluminum oxide), tin oxide, and the like. More preferably, the topical composition of the present invention comprises at least one inorganic sunscreen agent, such as titanium dioxide and zinc oxide, and most preferably, it comprises a blend of titanium dioxide and zinc oxide.

The average particle size of the inorganic particulate materials is preferably less than 1 micron, more preferably ranging from about 0.001 micron to about 0.1 micron, and most preferably from about 0.01 to about 0.1 micron. The inorganic particulate materials of the present invention can be of any regular or irregular shape, such as, for example, spherical, cubic, cylindrical, planar, fibrous, and the like. Further, such inorganic particulate materials may be surface-treated or coated with one or more layers of coating materials to impart desirable surface properties thereto. For example, the inorganic particulate materials may be coated with a hydrophilic or hydrophobic polymer, or encapsulated/entrapped in a hydrophilic or hydrophobic polymeric matrix, to improve the compatibility of such inorganic particulate materials with the carrier medium.

If present, the inorganic particulate material is in an amount ranging from about 0.1% to about 90%, preferably from about 5% to about 50%, and more preferably from about 10% to about 30%, by total weight of the composition.

The alkoxylated diphenylacrylate compound, the aryl carboxylic ester, and optionally one or more organic or inorganic sunscreen agents as described hereinabove can be added directly to any pharmaceutically or cosmetically acceptable carrier to form a cosmetic or topical composition. For purpose of the present invention, pharmaceutically or cosmetically acceptable carriers are substances that are biologically compatible with human skin and can be used to formulate active ingredients described hereinabove and/or hereinafter into a cream, gel, emulsion, liquid, suspension, powder, nail coating, skin oil, or lotion that can be topically applied. In the event that the cosmetically acceptable carrier is in the form of an emulsion, it may contain from about 0.1 to 99%, preferably from about 0.5 to 95%, more preferably from about 1 to 80% by weight of the total composition of water and from about 0.1 to 99%, preferably from about 0.1 to 80%, more preferably from about 0.5 to 75% by weight of the total composition of oil. In the event that the cosmetically acceptable carrier is in the form of an aqueous based gel, solution, or suspension, it may comprise from about 0.1 to 99 wt % of water and from about 0.1 to 75 wt % of other ingredients such as botanicals, non-aqueous solvents, etc.

Suitable components of the pharmaceutically or cosmetically acceptable carrier include, but are not limited to: moisturizing agents, astringent agents, chelating agents, emulsifiers or surfactants, sequestrants, emollients, preservatives, stabilizers, abrasives, adsorbents, thickeners, gellants, solidifying/structuring agents, anti-caking agents, anti-foaming agents, pH buffering/adjusting agents, binders, film formers, humectants, pigments, opacifiers, essential oils, fragrances, and aromatic compounds. The pharmaceutically or cosmetically acceptable carrier or carriers can be present in the topical or cosmetic composition of the present invention at an amount ranging from about 1% to about 99.9%, preferably from about 50% to about 99.5%, more preferably from about 70% to about 99%, and most preferably from about 80% to 90% by total weight of the topical or cosmetic composition.

The topical or cosmetic composition may contain one or more skin care additives, which are agents that provide benefits to the skin, rather than merely improving the physical or aesthetic characteristics of the topical composition. If present, such skin care actives may range from about 0.01 to 50%, preferably from about 0.05 to 35% by weight of the total composition. Exemplary skin care additives that can be used in the topical or cosmetic compositions of the present invention include, but are not limited to: self-tanning agents (e.g. dihydroxyacetone), anti-aging agents, DNA-repair enzymes, anti-wrinkle agents, anti-acne agents (e.g., resorcinol, salicylic acid, and the like), enzyme-inhibiting agents, collagen-stimulating agents, agents for the eradication of age spots and keratoses, analgesics, anesthetics, antimicrobials (e.g., antibacterials, antiyeast agents, antifungal agents, and antiviral agents), antidandruff agents, antidermatitis agents, antipruritic agents, antiemetics, anti-inflammatory agents, antihyperkeratolytic agents, antiperspirants, antipsoriatic agents, antiseborrheic agents, antihistamine agents, skin lightening agents, depigmenting agents, skin soothing/healing agents (e.g., aloe vera extract, allantoin, and the like), corticosteroids, hormones, antioxidants, proteins or peptides, vitamins and derivatives thereof (e.g., vitamin A, vitamin E, vitamin B₃, vitamin B₅, and the like), exfoliants, retinoids (e.g., retinoic acid and retinol), farnesol, bisabolol, phytantriol, glycerol, urea, guanidine (e.g., amino guanidine), clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, minocycline, hydroquinone, naproxen, ibuprofen, theophylline, cromolyn, albuterol, topical steroids (e.g., hydrocortisone, hydrocortisone 21-acetate, hydrocortisone 17-valerate, and hydrocortisone 17-butyrate), betamethasone valerate, betamethasone diproprionate, benzoyl peroxide, crotamiton, propranolol, promethazine, and mixtures or derivatives thereof. In a preferred, but not necessary embodiment of the present invention, the topical composition comprises one or more skin care actives selected from the group consisting of self-tanning agents, anti-aging agents, DNA repair enzymes, anti-wrinkle agents, anti-acne agents, antimicrobials, anti-inflammatory agents, skin-lightening agents, antioxidants, proteins or peptides, vitamins and derivatives thereof, exfoliants, and mixtures thereof.

For example, the topical or cosmetic compositions of the present invention may include one or more antioxidants, and more preferably one or more water-soluble extracts of biological materials that exhibit anti-oxidant activities. If present, such antioxidants or water-soluble extracts with anti-oxidant activities may range from about 0.01 to 45%, preferably from about 0.05 to 20%, more preferably from about 0.1 to 15% by weight of the total composition. Examples of suitable water-soluble extracts that exhibit anti-oxidant activities include, but are not limited to, extracts from: artemia, phytosphingosine, polygonum cuspidatum root, yeast such as saccharomyces lysate, thermos thermophillus ferment, birch (Betula alba), mimosa tenuiflora (bark) extract, fruit, clove, rye, malt, corn, spelt, millet, barley, oat, wheat, sesame, cumin, turmeric, green onion, celery, ginseng, ginger, licorice, carrot, bupleurum root, Ginkgo biloba (gingko), Foeniculi Fructus (fennel), kiwi, berry such as Morus bombycis (mulberry), Gentiana lutea (gentian), algae such as red algae, Arctium lappa (burdock), Salvia officinalis (sage), Lentinus edodes (shiitake mushroom), Perilla frutescens (perilla), Filipendula Multijuga, Fucus vesiculosis (bladderwrack, sea weed), peach kernel, Allium sativum (garlic), Poria cocos (poria), Humulus lupulus (hops), Mutan Cortex (Moutan Bark), Pimpinella major, Lactuca sative (lettuce), Astragalus membranaceous (astragalus) and Rosmarinus officinalis (rosemary), Prunus amygdalus (almond), Althea officinale (althea), aloe, Rosae Fructus (rose fruit, or Rosa multiflora), Scuttelaria baicalensis (Huang qin), Puerariae Radix (Pueraria Root, or Pueraria lobata), chamomile such as Chamomillae Flos (German chamomile), Gardenia jasminoides (zhii zi, Gardeniae Fructus), Sophora flavescens Aiton (Sophorae Radix), chlorella, rice bran, Paeoniae lactiflora (white peony), ziyu (Sanguisorba officinalis, burnet), Morus alba (sang bai pi, mulberry), Glycine max (soybean), Camellia sinensis (tea), Carthami Flos (safflower), Aesculus hippocastanum (horse chestnut), Melissa officinalis (lemon balm) and Coicis Semen (Coix lacryma-jobi var. ma-yuen), Angelica keisukei, Arnica montana (arnica), Foeniculum officinale (fennel), Isodon japonicus Hara (Isodonis Herba), Daucus Carota (carrot), Oryza sativa (rice), Crataegus cuneata (Japanese howthorn), Acores calamus (sweet flag), Crataegus oxycantha (howthorn), Juniperus communis, Ligusticum wallichii (Chinese lovage), Swertiae Herba (Swertia Herb), Thymus vulgaris (garden thyme), Citrus reticulata (Citrus unshiu), Capsicum tincture, Angelicae sinensis (angelica), Aurantii Pericarpium (bitter orange peel), Ruscus aculeatus (butcher's bloom), Vitis vinifera (grape), Tilia japonica (lime), Citrus junos and Rosa canina (rose hip), caffeine, Cinnamomi Cortex (cinnamon bark) and Eriobotrya japonica Lindl. (loquat), Gambir, Echinacea, Phellodendri Cortex (amur cork tree or Phellodendron amurense), Hypericum perforatum (St. John's wort), Citrus sinensis (orange), Valeriana fauriei Briquet, Artemisia capillaris Thunb., Cucumis sativus (cucumber), Geranii Herba (Geranium Herb), Lithospermum erythrorhizon Sieb. et Zucc., Hedera helix, Achillea millefolium (yarrow), Ziziphus jujuba (Chinese dates), Calendula officinalis (pot marigold), Houttuynia cordata (Houttuyniae Herba, Houttuynia Herba), Potentilla erecta, Petroselinum crispum (parsley), Parietaria officinalis, Santalum album (sandalwood), Prunus persica (peach), Centaurea cyanus (cornflower), Eucalyptus globulus (eucalyptus) and Lavandula angustifolia (lavender), Persea americana (avocado), Nasturtium officinalis (watercress), Symphytum officinale (comfrey), Asarum sieboldii (wild ginger), Xanthoxyum piperitum (Japan pepper), Rehmannia glutinosa (di huang), Mentha piperita (peppermint), Syzygium aromaticum (clove), Tussilago farfara (coltsfoot) and Haematoxylum campechianum (logwood); Oolong tea, Cinchona succirubra (peruvian bark), Betula verrucosa (birch) and Glechoma hederacea (ground ivy), milk and royal jelly, honey, cysteine and derivatives thereof, ascorbic acid and derivatives thereof, BHA, BHT, ferulic acid and derivatives thereof, grapeseed extract, pine bark extract, horseradish extract, hydroquinones, rosmarinic acid, coffee robusta seed, caffeic acid, tocopherol and derivatives thereof, green tea extract, sodium DNA, sodium ribonucleic acid, octyl, propyl and dodecyl gallates, uric acid and thiodiproprionate derivatives.

For another example, the topical or cosmetic compositions of the present invention may include one or more DNA repair enzymes and more preferably one or more DNA repair enzymes selected from the group consisting of 8-oxoguanine DNA glucosylase, uracil-and-hypoxanthine-DNA-glycosylase, damaged-base glycosylase (e.g., 3-methyl-ladenine-DNA glycosylase), 3-methyladenine-DNA-glycosylase, pyrimidine dimer-specific glycosylase, pyrimidine glycosylase/abasic lyase, N-glycosylase/apyrimidinic lyase, N-glycosylase/apurinic-apyrimidinic lyase, photolyase, O⁶-methylguanine-DNA-methyl transferase, T4 endonuclease V, pyrimidine dimer-specific endonuclease, apyrimidin/apurin-endonuclease, UV damage endonuclease, correndonuclease, and DNA exonuclease. Other DNA repair enzymes or enzyme complexes involved in either the base excision repair (BER) pathway, the nucleotide excision repair (NER) pathway, or the alternative excision repair pathway can also be used for practice of the present invention. Such DNA repair enzymes may be derived or extracted from suitable sources, such as bacteria, algae, protozoans, planktons, plants, and the like. Further, the DNA repair enzymes may be encapsulated in liposomes for more efficient delivery to the skin. Liposomes are microscopic vesicles consisting of an aqueous core enclosed in one or more lipid layers formed by membrane lipids, such as phospholipids and sphingomyelins. Liposomes facilitate transfer of skin care actives into the dermis of skin. For more details regarding encapsulation of DNA repair enzymes in liposomes, see U.S. Pat. No. 5,296,231, the contents of which are incorporated herein by reference in their entireties for all purposes. If present, the DNA repair enzymes may range from about 0.01% to 20%, preferably from about 0.1% to about 10%, and more preferably desirably from about 0.5% to about 2%, by total weight of the composition.

The cosmetically acceptable carrier may also contain one or more oils, which are also known as skin conditioning agents, such as volatile or nonvolatile silicones, esters, paraffinic hydrocarbons, vegetable oils, and synthetic oils. Suitable volatile or non-volatile silicones include, but are not limited to: cyclomethicone; methyl trimethicone; octamethyltrisiloxane; decamethyltetrasiloxane; dodecamethylpentasiloxane; dimethicone; phenyl trimethicone trimethylsiloxyphenyl dimethicone; phenyl dimethicone; cetyl dimethicone; dimethicone copolyol, cetyl dimethicone copolyol; glycerolated silicones such as lauryl PEG-9 polydimethylsiloxyethyl dimethicone; or mixtures thereof. In one embodiment, the composition may contain one or more non-volatile silicone oils having a viscosity ranging from about 5 to 250,000 cst at 25° C. Examples include dimethicone, phenyl trimethicone, diphenyl dimethicone, and the like. Suitable esters include mono-, di-, or triesters.

Monoesters are in the general form RCO—R′ wherein R and R′ are each independently a C₁₋₄₅ straight or branched chain, saturated or unsaturated alkyl. Diesters may be formed by the reaction of a C₁₋₄₅ aliphatic or aromatic mono- or dihydric alcohol with a C₁₋₄₅ aliphatic or aromatic mono- or dicarboxylic acid, as appropriate, where the aliphatic group may be straight or branched chain, or saturated or unsaturated. Suitable triesters include the reaction products of a C₁₋₄₅ aliphatic or aromatic alcohol having at least three hydroxyl groups with a C₁₋₄₅ carboxylic acid, or a C₁₋₄₅ aliphatic or aromatic alcohol with a C₁₋₄₅ tricarboxylic acid, with the aliphatic chains being linear or branched, saturated or unsaturated. Examples include esters of caprylic and capric acids and glycerin such as caprylic/capric triglycerides; esters of glycerin or polyglycerin and stearic acid such as glyceryl stearate, diglyceryl diisostearate; esters of malic acid and isostaryl alcohol such as diisostearyl malate; coco caprylate caprate and the like. If present, such oils may range from about 0.1 to 99% by total weight of the composition.

The cosmetically acceptable carrier may further contain one or more emulsifiers or surfactants. Preferably, it contains one or more nonionic organic emulsifiers. Nonionic organic emulsifiers suitable for the practice of the present invention include alkylene-oxide-derived emulsifiers. For example, one group of such alkylene oxide-derived emulsifiers can be formed by reaction of an alcohol with an alkylene oxide, such as ethylene or propylene oxide. Preferably the alcohol is a fatty alcohol having 6 to 30 carbon atoms. This group of emulsifiers have the general formula of R(X)nOR′, wherein R is a C₆-C₃₀ alkyl group, X is either —OCH₂CH₂— or —OCH₂CH(CH₃)— (as derived from ethylene oxide or propylene oxide), n is an integer from about 6 to about 200, and R′ is either hydrogen or a C₆-C₃₀ alkyl group. Examples for this group of emulsifiers include Steareth 2-100, which is formed by the reaction of Ceteth 1-45 which is formed by the reaction of cetyl alcohol and ethylene oxide, and the number of repeating ethylene oxide units is 1 to 45; stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; and the like. Another group of the alkylene oxide-derived emulsifiers can be formed by reaction of alkylene oxides with fatty acids (i.e. alkylene oxide esters of fatty acids). This group of emulsifiers have the general formula of RCO(X)nOH or RCO(X)nOOCR, wherein R is a C₆-C₃₀ alkyl group, X is either —OCH₂CH₂— or —OCH₂CH(CH₃)— (as derived from ethylene oxide or propylene oxide), and n is an integer from about 6 to about 200. Examples for this group of emulsifiers include PEG-6 stearate, PEG-10 stearate, PEG-12 stearate, PEG-100 stearate, PEG-8 dilaurate, PEG-10 distearate, and the like. Yet another group of the alkylene oxide-derived emulsifiers can be formed by reaction of alkylene oxides with both fatty acids and fatty alcohols (i.e., wherein the polyalkylene oxide portion is esterified on one end with a fatty acid and etherified on the other end with a fatty alcohol). This group of alkylene oxide-derived emulsifiers have the general formula RCO(X)nOR′, wherein R and R′ are C₆-C₃₀ alkyl groups, X is either —OCH₂CH₂— or —OCH₂CH(CH₃)— (as derived from ethylene oxide or propylene oxide), and n is an integer from about 6 to about 200. Still a further group of the alkylene oxide-derived emulsifiers can be formed by reaction of alkylene oxides with fatty acids and mono-, di- or polyhydric alcohols, including but not limited to glycerin or sugars (such as glucose, galactose, methyl glucose). Examples for this group of emulsifiers include PEG glyceryl oleates, PEG glyceryl stearate, and PEG polyhydroxyalkanotes, such as PEG dipolyhydroxystearate wherein the number of repeating ethylene glycol units ranges from 3 to 1000. Homopolymeric or block copolymeric ethers formed by the polymerization of monomeric alkylene oxides (e.g., ethylene or propylene oxide) are also suitable as nonionic emulsifiers for the practice of the present invention.

Condensation products of fatty alcohols with sugar or starch polymers can also be used as nonionic emulsifiers in the present invention. This group of emulsifiers can be represented by the formula of (S)nOR, wherein S is a sugar moiety such as glucose, fructose, mannose, and galactose, n is an integer of from about 1 to about 1000, and R is a C₆-C₃₀ alkyl group. Preferred examples of this group of emulsifiers include decyl polyglucoside (commercially available as APG 325 CS from Henkel) and lauryl polyglucoside (commercially available as APG 600 CS and 625 CS from Henkel).

Other suitable nonionic emulsifiers include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkoxylated sorbitan can be esterified with C₆₋₃₀, preferably C12-22 fatty acids. Examples of this group of emulsifiers include Polysorbates 20-85, sorbitan oleate, sorbitan sesquioleate, sorbitan palmitate, sorbitan sesquiisostearate, sorbitan stearate, and the like.

Also suitable are phosphate-based emulsifiers such as those which are C₂₋₄₀ alcohols and phosphoric acid. More preferred is where the alcohols are fatty C6-22 alcohols such as cetyl, stearyl, behenyl, alcohols or salts thereof. Also suitable are diesters phosphoric acid and one or more C2-40 alcohols, e.g. dicetyl phosphate; or fatty C6-30 ethoxylated alcohols and phosphoric acid, e.g. ceteth-10 phosphate, steareth-10 phosphate; C12-13 Pareth-2 Phosphate; Laureth-7 Phosphate; and the like.

Inulin based emulsifiers may also be used. Examples include inulin lauryl carbamate; palmitoyl inulin; sodium carboxymethyl inulin; stearoyl inulin; undecylenoyl inulin; and mixtures thereof.

It may also be desirable to incorporate various types of surfactants that induce the formation of liquid crystals in the composition. Examples of such surfactants include ceramide based surfactants such as phytosphingosines; C12-20 alkyl glucosides; coco glucosides; olive based semi-solids such as cetyl palmitate; sorbitan olivate; sorbitan palmitate; or phospholipid based emulsifiers such as lecithin, hydrogenated lecithin, phytosterols, and so on.

Preferred among the nonionic emulsifiers described hereinabove are those selected from the group consisting of steareth-2, steareth-21, steareth-100, ceteareth-20, ceteareth-12, PEG-6 stearate, PEG-10 stearate, PEG-12 stearate, PEG-100 stearate, and mixtures thereof.

The nonionic emulsifier may present in the topical composition of the present invention in a total amount ranging from about 0.1% to about 50%, preferably from about 1% to about 40%, and more preferably from about 2% to about 20%, by total weight of the composition. All percentages mentioned herein are percentages by weight unless otherwise indicated.

The cosmetically acceptable carrier may also comprise one or more humectants, which include, but are not limited to: glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and the like. Preferably, the humectants used in the composition of the invention are C₁₋₆, preferably C₂₋₄ alkylene glycols, most particularly butylene glycol, or glycerin. If present, such humectants may range from about 0.001% to about 25%, preferably from about 0.005% to about 20%, more preferably from about 0.1% to about 15%, by total weight of the topical composition.

The cosmetically acceptable carrier may also comprise one or more organosiloxane elastomers, generally those known as non-emulsifying. If present, such elastomers may range from about 0.1 to 30% by weight of the total composition. Examples of suitable elastomers include, but are not limited to dimethicone/vinyl dimethicone crosspolymer, methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl(3,3,-trifluoropropyl)siloxane copolymers, and the like.

The compositions of the invention may also contain other ingredients such as structuring agents in the form of polymeric structuring agents such as acrylic polymers, polyamides or polyurethanes. The structuring agents may be water or oil soluble or dispersible. Such structuring agents will provide structure, or increase the viscosity of the composition. If present, suggested ranges are from about 0.1 to 50%, preferably from about 0.5 to 40%, more preferably from about 1 to 35% by weight of the total composition. Suitable structuring agents include natural, synthetic waxes, or mineral waxes such as petrolatum, candelilla, ozokerite, synthetic wax, polyethylene, silicone waxes such as stearyl or behenyl dimethicone, and the like. Suitable polymeric structuring agents include carbomer, acrylic polymers or copolymers, such as acrylates copolymer, polyacrylate-1 crosspolymer, acrylates/C10-30 alkyl acrylate crosspolymer, C10-30 alkyl acrylate crosspolymer (e.g., those commercially available as Carbopol® or Pemulen®), ester or amide terminated polyamides (those commercially available from Arizona Chemical under the Uniclear® or Sylvaclear®), or aqueous dispersions or solutions of polyurethanes.

The topical compositions of the present invention, especially the sunscreen compositions, are preferably water-resistant, sweat-proof, non-irritating, non-comedogenic (i.e., will not clog pores), and/or hypo allergenic. More preferably, the topical compositions are transparent or translucent, non-whitening, and can be easily absorbed into the skin. Most preferably, the topical compositions provide maximum, broad spectrum photoprotection and a therapeutic or curative benefit for sun-damaged or photosensitive skin and will have an SPF ranging from about 15 to 100, preferably from about 20 to 75.

The topical compositions of the present invention are designed so that they may be worn daily and may be worn under make-up. Because in one embodiment of the invention the topical compositions of the present invention may contain relatively lower concentrations of organic chemical sunscreen agents, they are often perceived as more desirable to consumers who prefer sunscreens that do not have organic sunscreen compounds in them. Preferably, the compositions may be topically applied to any portion of the skin that will be or will tend to be exposed to UV irradiation, including, but not limited to, the face, the ears, the scalp, the hands, arms, shoulders, legs, feet, abdomen and back, and any area of the skin that an individual chooses to expose to UV, visible, and/or IR irradiation. Such UV irradiation is typically, but not necessarily, directed to the skin from the sun. Other UV, visible, and IR range light sources include most typical UV light sources, as will be appreciated by those of skill in the art, and also including most industrial light sources.

### EXAMPLES

The following examples are illustrative, but not limiting, of the topical compositions of the present invention as described hereinabove.

### Example 1

An anhydrous topical sunscreen composition containing Methoxycrylene, 2-phenyethyl benzoate, and Avobenzone, can be prepared as follows:

Composition I

| Ingredients | Wt % |
|---|---|
| Dipentaerythrityl hexahydroxystearate/hexastearate/hexarosinate | 1.49 |
| Shea butter | 2.00 |
| Isononyl isononanoate | 1.00 |
| Polyglyceryl-2 triisostearate | 2.81 |
| Heptyl undecylenate | QS |
| Microcrystalline wax | 4.50 |
| Synthetic wax/synthetic beeswax/stearic acid | 2.40 |
| Polyethylene | 6.00 |
| Beeswax | 1.80 |
| 2-phenyethyl benzoate | 6.00 |
| Bis-diglyceryl polyacyladipate-2 | 5.29 |
| Avobenzone | 3.00 |
| Ethylhexyl salicylate | 5.00 |
| Homosalate | 5.00 |
| VP/eicosene copolymer | 1.00 |
| Ethyl macadamiate | 2.00 |
| HDI/trimethylol hexyllactone crosspolymer//silica | 3.50 |
| Mica/methyl methacrylate crosspolymer | 5.00 |
| Mica/aluminum dimyristate/triethoxycaprylylsilane/disodium | 5.00 |
| stearoyl glutamate | |
| Glyceryl dilaurate | 1.00 |
| Oleic/linoleic/linolenic polyglyceride | 4.00 |
| Glyceryl hydrogenated rosinate | 4.00 |
| Tricaprylin | 1.31 |
| Jojoba seed oil | 4.00 |
| Mauritia flexuosa fruit oil | 1.00 |
| Methyl glucose sesquistearate | 1.50 |
| Methoxycrylene | 6.00 |
| Pentaerythrityl tetra-di-t-butyl hydroxyhydrocinnamate | 0.01 |
| Tocopheryl acetate | 0.10 |
| Ethylhexyl methoxycinnmate | 7.50 |

### Example 2

Two anhydrous topical sunscreen composition containing Methoxycrylene, 2-phenyethyl benzoate, and Avobenzone can be prepared as follows:

Compositions II and III

| | Wt % | |
|---|---|---|
| Ingredients | Comp. I | Comp. II |
| Dipentaerythrityl | 1.49 | 1.49 |
| hexahydroxystearate/hexastearate/hexarosinate | | |
| Shea butter | 2.00 | 2.00 |
| Isononyl isononanoate | 1.00 | 1.00 |
| Polyglyceryl-2 triisostearate | 2.81 | 2.81 |
| Moringa oleifera seed oil | QS | QS |
| Microcrystalline wax | 4.50 | 4.50 |
| Synthetic wax/synthetic beeswax/stearic acid | 2.40 | 2.40 |
| Polyethylene | 6.00 | 6.00 |
| Beeswax | 1.80 | 1.80 |
| 2-phenyethyl benzoate | 6.00 | 5.00 |
| Bis-diglyceryl polyacyladipate-2 | 8.50 | 9.79 |
| Avobenzone | 3.00 | 3.00 |
| Ethylhexyl salicylate | 5.00 | 5.00 |
| Homosalate | 5.00 | 5.00 |
| VP/eicosene copolymer | 1.00 | 1.00 |
| Ethyl macadamiate | 2.00 | 2.00 |
| HDI/trimethylol hexyllactone crosspolymer//silica | 3.50 | 3.50 |
| Mica/methyl methacrylate crosspolymer | 5.00 | 5.00 |
| Mica/aluminum | 5.00 | 5.00 |
| dimyristate/triethoxycaprylylsilane/disodium | | |
| stearoyl glutamate | | |
| Glyceryl dilaurate | 1.00 | 1.00 |
| Oleic/linoleic/linolenic polyglyceride | 4.00 | 4.00 |
| Glyceryl hydrogenated rosinate | 4.00 | 4.00 |
| Tricaprylin | 1.31 | 1.31 |
| Jojoba seed oil | 4.00 | 4.00 |
| Mauritia flexuosa fruit oil | 1.00 | 1.00 |
| Methyl glucose sesquistearate | 1.50 | 1.50 |
| Methoxycrylene | 2.79 | 3.00 |
| Pentaerythrityl tetra-di-t-butyl | 0.01 | 0.01 |
| hydroxyhydrocinnamate | | |
| Tocopheryl acetate | 0.10 | 0.10 |
| Ethylhexyl methoxycinnmate | 7.50 | 5.00 |

### Example 3

Two water-in-silicone emulsion-based face lotions with SPF containing Methoxycrylene and 2-phenyethyl benzoate can be prepared as follows:

Compositions IV and V

| | Wt % | |
|---|---|---|
| | Comp. | Comp. |
| Ingredients | IV | V |
| Neopentyl glycol diethylhexanoate | 3.46 | 3.46 |
| Ethyl macadamiate | 2.00 | 2.00 |
| Beeswax | 3.00 | 3.00 |
| Polydiethylsiloxane | 3.00 | 3.00 |
| Zinc oxide/C₁₂-C₁₅ alkyl benzoate/isostearic | 10.00 | 10.00 |
| acid/polyhydroxystearic acid | | |
| Cetyl PEG/PPG-10/1 dimethicone | 1.00 | 1.00 |
| Dimethicone//dimethicone PEG-10/15 crosspolymer | 2.00 | 2.00 |
| Ethylhexyl palmitate/quaternium-90 | 2.00 | 2.50 |
| bentonite/dimethicone/propylene carbonate | | |
| Lauryl PEG-9 polydimethylsiloxyethyl dimethicone | 2.00 | 2.00 |
| Dimethicone/vinyl dimethicone crosspolymer//methyl | 1.50 | 150 |
| trimethicone | | |
| Titanium dioxide/dimethicone | 4.00 | 4.00 |
| Methyl trimethicone | 11.25 | 11.25 |
| Dimethicone/trimethylsiloxysilicate | 1.00 | 1.00 |
| Dimethicone | 1.50 | 1.50 |
| Phenoxyethanol | 0.20 | 0.20 |
| Pantethine | 0.20 | 0.20 |
| Tocopheryl acetate | 0.20 | 0.20 |
| 2-phenyethyl benzoate | 3.00 | 6.00 |
| Methoxycrylene | 5.00 | 6.00 |
| Deionized water | QS | QS |
| Magnesium sulfate | 1.00 | 1.00 |
| Dipentaerythrityl tri-polyhydroxystearate | 2.00 | 2.00 |
| Glycerin | 2.00 | 2.00 |
| Phenoxyethanol/caprylyl glycol | 1.10 | 1.10 |
| Butylene glycol | 6.00 | 6.00 |
| Xanthan gum | 0.25 | 0.25 |
| Iron oxides | 0.50 | 0.50 |
| Lecithin | 0.01 | 0.01 |

### Example 4

Two oil-in-water emulsion-based sunscreen compositions with SPF containing Methoxycrylene and 2-phenyethyl benzoate can be prepared as follows:

Compositions VI and VII

| | Wt % | |
|---|---|---|
| | | Comp. |
| Ingredients | Comp. VI | VII |
| Deionized water | QS | QS |
| Disodium EDTA | 0.10 | 0.10 |
| Caprylyl glycol | 0.30 | 0.30 |
| Butylene glycol | 4.00 | 4.00 |
| Xanthan gum | 0.30 | 0.30 |
| Avobenzone | 3.00 | 3.00 |
| Ethylhexyl methoxycinnamate | 5.00 | 5.00 |
| Shea butter | 3.00 | 3.00 |
| Ptassium cetyl phosphate | 1.00 | 1.00 |
| PEG-100 stearate | 2.25 | 2.25 |
| 2-phenylethyl benzoate | 5.00 | 5.00 |
| Glyceryl stearate | 1.50 | 1.50 |
| Cetyl alcohol | 0.50 | 0.50 |
| Methoxycrylene | 3.00 | 3.00 |
| Styrene/acrylates copolymer//PEG-8 laurate//water | 1.00 | 1.00 |
| Ethylhexyl salicylate | — | 5.00 |
| Homosalate | — | 5.00 |
| Lauryl PEG-9 polydimethylsiloxyethyl dimethicone | 2.00 | 2.00 |
| Methyl trimethicone | 2.00 | 2.00 |
| Dimethicone | 1.00 | 1.00 |
| Ethylhexylglycerin | 0.50 | 0.50 |
| Phenoxyethanol/caprylyl glycol | 0.85 | 0.85 |

While some illustrative embodiments of the inventions have been described hereinabove, such illustrative embodiments should not be interpreted in any manner to limit the broad scope of the prevent invention. Various modifications and equivalents of the described embodiments and components thereof will be apparent to those of ordinary skill in the art. Some modifications and equivalents will be readily recognized by one ordinarily skilled in the art, while others may require no more than routine experimentation. It is therefore understood that such modifications and equivalents are within the spirit and scope of the present invention.

What is claimed is:

1. A topical composition comprising an alkoxylated diphenylacrylate compound and an aryl carboxylic ester in a pharmaceutically or cosmetically acceptable carrier.

2. The topical composition of claim 1, wherein the alkoxylated diphenylacrylate compound is an alkoxylated α-cyanodiphenylacrylate.

3. The topical composition of claim 2, wherein the alkoxylated α-cyanodiphenylacrylate compound has the formula (I): 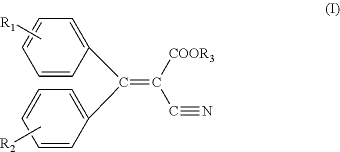
wherein one or both of R₁ and R₂ is a straight or branched-chain C₁-C₃₀ alkoxy radical, any non-alkoxy radical R₁ or R₂ is hydrogen, and R₃ is a straight or branched-chain C₁-C₃₀ alkyl radical.

4. The topical composition of claim 3, wherein one or both of R₁ and R₂ is a straight or branched-chain C₁-C₈ alkoxy radical.

5. The composition of claim 2, wherein the alkoxylated α-cyanodiphenylacrylate compound has the formula (IV), (V) or (VI): 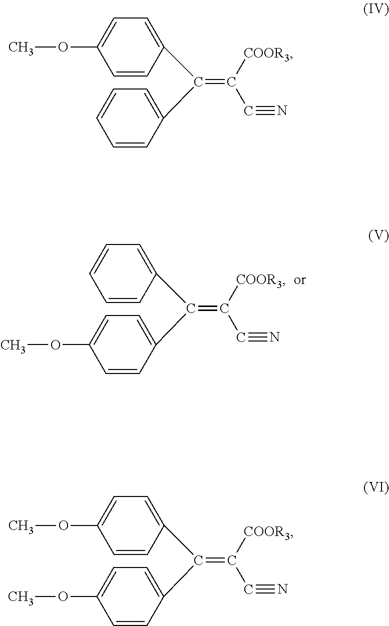
wherein R₃ is a straight or branched-chain C₁-C₃₀ alkyl radical.

6. The topical composition of claim 2, wherein the alkoxylated α-cyanodiphenylacrylate compound is α-ethylhexyl α-cyano-β-(4-methoxyphenyl)-β-phenylacrylate.

7. The topical composition of claim 1, wherein the aryl carboxylic ester is an ester of an aryl carboxylic acid and an arylalkyl alcohol.

8. The topical composition of claim 1, wherein the aryl carboxylic ester has the formula (II): 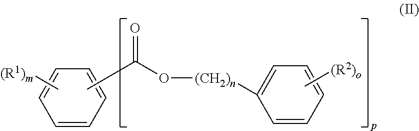
wherein R¹ and R² may be identical or different and are selected from the group consisting of a hydroxy group, a linear or branched C₁-C₄ alkyl or alkoxy radical, a nitro radical, an amino radical, and a C₆H₆SO₂ radical; m is an integer ranging from 0 to 5; n is an integer ranging from 1 to 10; o is an integer ranging from 0 to 5; p is an integer ranging from 1 to 6, provided that m+p<=6.

9. The topical composition of claim 1, wherein the aryl carboxylic ester is selected from the group consisting of 2-phenylethyl benzoate, 2-phenylethyl toluate, di-2-phenylether phthalate.

10. The topical composition of claim 1, further comprising a dibenzoylmethane UV-screening agent.

11. The topical composition of claim 10, wherein the dibenzoylmethane UV-screening agent comprises 4,4′-t-butyl methoxydibenzoylmethane.

12. The topical composition of claim 11, further comprising one or more additional organic sunscreen agents selected from the group consisting of 2-ethylhexylsalicylate, 3,3,5-trimethylcyclohexylsalicylate, 2-ethylhexyl p-methoxycinnamate, 2-hydroxy-4-methoxybenzophenone, 2,2-dihydroxy-4-methoxybenzophenone, 2,4-bis-{4-(2-ethyl-hexyloxy)-2-hydroxyl-phenyl}-6-(4-methoxyphenyl)-1,3,5-triazine, methylene bis-benzotriazolyl tetramethylbutylphenol, terephthalylidene dicamphor sulfonic acid, diethylhexyl 2,6-naphthalate, digalloyltrioleate, ethyl 4-[bis(hydroxypropyl)]aminobenzoate, glycerol p-aminobenzoate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid, and mixtures or combinations thereof.

13. The topical composition of claim 1, further comprising at least one physical sunscreen agent selected from the group consisting of titanium dioxide, zinc oxide, and mixtures thereof.

14. The topical composition of claim 1, further comprising at least one DNA repair enzyme.

15. The topical composition of claim 1, further comprising glycerin or a C₂₋₄ alkylene glycol.

16. A topical composition having an SPF ranging from about 15 to 100 comprising an alkoxylated diphenylacrylate compound and an aryl carboxylic ester in a pharmaceutically or cosmetically acceptable carrier.

17. The topical composition of claim 16, wherein the an alkoxylated diphenylacrylate compound is α-ethylhexyl α-cyano-β-(4-methoxyphenyl)-β-phenylacrylate, and wherein the aryl carboxylic ester is 2-phenylethyl benzoate.

18. The topical composition of claim 16, further comprising 4,4′-t-butyl methoxydibenzoylmethane.

19. The topical composition of claim 16, further comprising a physical sunscreen agent selected from the group consisting of zinc oxide particles, titanium dioxide particles, and mixtures thereof.

20. The topical composition of claim 16, further comprising at least one nonvolatile silicone having a viscosity ranging from about 5 to 250,000 centistokes at 25° C. and at least one silicone elastomer.
